(19) United States
(12) Patent Application Publication
(10) Pub. No.: US 2025/0059123 A1
(43) Pub. Date: Feb. 20, 2025

(54) PROCESS FOR THE PREPARATION OF DIKETONES AND PYRROLE DERIVATIVES

(71) Applicant: Pirelli Tyre S.p.A., Milano (IT)

(72) Inventors: Luca GIANNINI, Milano (IT); Maurizio Stefano GALIMBERTI, Milano (IT); Vincenzina BARBERA, Milano (IT); Simone NADDEO, Milano (IT)

(73) Assignee: Pirelli Tyre S.p.A., Milano (IT)

(21) Appl. No.: 18/720,357

(22) PCT Filed: Dec. 19, 2022
(86) PCT No.: PCT/IB2022/062453

§ 371 Date: Jun. 14, 2024

(30) Foreign Application Priority Data
Dec. 22, 2021 (IT) 102021000032138

(51) Int. Cl. C07C 45/59; C07D 207/325

(52) U.S. Cl. CPC C07C 45/59; C07D 207/325

(57) ABSTRACT

The present invention relates to a process for the preparation of diketones and pyrrole derivatives, optionally substituted in the 2 and/or 5 positions, from furans.

### PRIOR ART

The Applicants have long studied the formation of adducts between pyrrole derivatives and sp² hybridized carbon allotropes and their multiple uses, in particular as reinforcing fillers in elastomeric mixtures, for example for use in the production of tires for vehicles. See for example WO 2016/050887 and WO 2018/087685.

The Applicants have in fact demonstrated that it is possible to obtain stable adducts between pyrrole derivatives and sp² hybridized carbon allotropes by exploiting the specific characteristics of such compounds, with various advantages obtainable through the use of these adducts in tires compounds.

The pyrrole derivatives are typically obtained by Knorr-Paal reaction between a diketone and a primary amine (see the scheme below). The reaction is performed at high temperatures, in presence or absence of solvent and/or of a catalyst and makes it possible to obtain a vast range of pyrrole derivatives in high yields. In this regard see for example WO2015/189411.

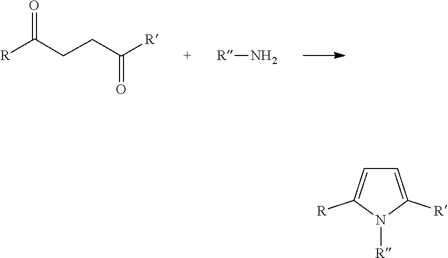

In particular, 2,5-hexanedione is often used as the starting diketone for obtaining pyrrole derivatives wherein the pyrrole ring is substituted at the 2 and 5 positions with a methyl group. This compound is moreover a reagent widely used in the chemical industry as a reaction intermediate for a vast range of synthetic processes, and its commercial value is very high.

In the art, various processes are known for the synthesis of diketones, and in particular of 2,5-hexanedione.

In ChemSusChem 2014, 7, 2089, Fei Liu et al. describe the synthesis of diketones from carbohydrates or furan derivatives by hydrogenation catalyzed by Pd/C in a CO₂ atmosphere (30-40 bar) at high temperature.

Similarly, Hu Li et al. (Green Chemistry, 2022, 22, 582) have described the synthesis reaction of 1-hydroxyhexane-2,5-dione by hydrogenation of 5-hydroxmethyl-furfural derived from biomass, in ethanol at 140° C. using an iridium complex as catalyst.

Various literature articles report acid-catalyzed ring opening reactions of 2,5-dimethylfuran (1) to obtain 2,5-hexanedione (2) (see scheme below): B. Kuhlmann et al. (Journal of Organic Chemistry 1994, 59, 3098-3101) describe the reaction in pure deuterium oxide at the temperature of 250° C.; Goldberg et al. (Journal of Molecular Catalysis 1989, 57, 91-103) use a cationic exchange resin as catalyst, performing the reaction at room temperature for more than 72 hours; in Green Chemistry 2016, 18, 220-225 the use of a cationic exchange resin in a mixture of water and tetrahydrofuran (1:9 by volume) at 90° C. is described; finally, in Advanced Materials Research 2012, 518, 3947-3950, the reaction is performed in water and glacial acetic acid at 85° C. in presence of 10% of sulfuric acid.

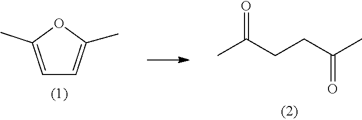

Similarly, in Catalysis Science & Technology 2013, 3, 106 the ring opening reaction is described on various furan derivatives, to obtain the corresponding diketones, performed in 1:1 mixtures of water and an organic solvent, at temperatures of 60-80° C., in the presence of 10 mol % of a strong acid.

As described for example in Chemistry Select 2016, 6, 1252-1255, the prolonged contact of the 2,5-hexanedione produced with the acids in the reaction medium leads to the formation of significant quantities of by-products of oligomerization. In this article, the authors propose the use of a two-phase system wherein the aqueous phase contains the dimethylfuran and the acidic catalyst (for example HCl, H₂SO₄ and H₃PO₄), while the organic phase (for example methyl isobutyl ketone) enables the continuous extraction of the 2,5-hexanedione produced.

Considerations on the mechanism of the hydrolysis reaction of furans, to obtain the corresponding diketones, have been reported for example by Nikbin Nima et al, in the literature article ChemSusChem 2013, 6(11), 2066-2068.

However, the methods described above are poorly suited for use on the industrial scale because of the high temperatures and reaction conditions used, the use of large quantities of organic solvents and the need for long and costly processing and/or purification processes at the end of the reaction.

### SUMMARY OF THE INVENTION

The Applicants set themselves the objective of developing a process for the synthesis of diketones capable of overcoming at least in part the disadvantages of the processes known in the art.

In particular, the Applicants set themselves the objective of developing an economically advantageous and eco-compatible process, easily applicable on the industrial scale, for obtaining diketones. It would further be particularly desirable that such a process would make it possible to obtain diketones immediately usable as intermediates for the synthesis of more complex molecules without the need for further processing or purification steps.

The Applicants have surprisingly found that these objectives can be attained carrying out the ring opening reaction on a furan, optionally substituted in the 2 and/or 5 positions, also at low temperatures (even at room temperature), in a controlled quantity of water with the addition of a suitable acid.

The present invention thus relates to a process for the preparation of a diketone of formula (II) according to the following scheme (1)

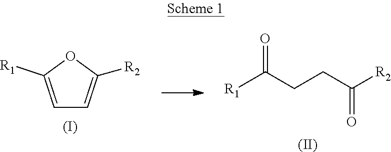

wherein R₁-R₂ are independently selected in the group consisting of hydrogen, a linear or branched C₁-C₁₈ alkyl group, a linear or branched C₂-C₁₈ alkenyl or alkynyl group, an alkyl-aryl group with linear or branched C₁-C₁₈ alkyl, an alkenyl-aryl group with linear or branched C₂-C₁₈ alkenyl, an alkynyl-aryl group with linear or branched C₂-C₁₈ alkynyl, aryl, and heteroaryl; wherein said process comprises the phases of:
a) preparing a mixture composed of: water, an organic or inorganic Brønsted-Lowry acid having a pKₐ value lower than 3.5 and the one or more conjugate bases whereof have a standard reduction potential value lower than 0.5 volts measured under acidic conditions, and a furan of formula (I); and
b) stirring the mixture, optionally heating;
and wherein in the mixture prepared in phase a) the molar ratio between water and furan of formula (I) is at least 1, preferably of from 1 to 4, and wherein said organic or inorganic Brønsted-Lowry acid is present in an amount corresponding to at least 2 mol % with respect to the amount of furan of formula (I).

In a further embodiment of the present invention, the process further comprises a second step wherein, after the phase b) as previously described, a primary amine R₃—NH₂ (III) is added, the mixture thus obtained is stirred, and optionally further heated, to obtain a pyrrole derivative of formula (IV) according to the following synthetic scheme (2)

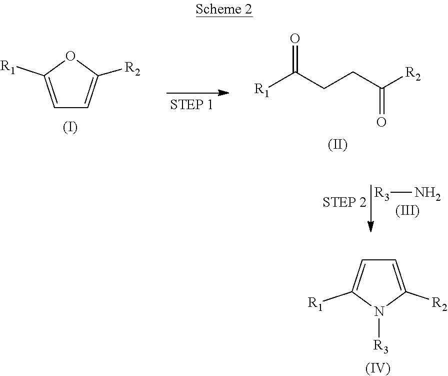

wherein R₁-R₂ are as previously defined and wherein R₃ is selected in the group consisting of:

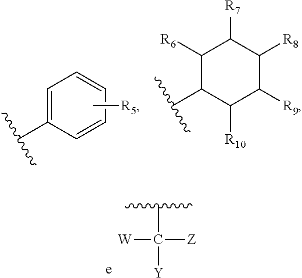

wherein
R₅ is hydrogen, alkyl, aryl, benzyl, amine, alkylamine, arylamine, benzylamine or aminoaryl;
R₆-R₁₀ are independently selected in the group consisting of: hydrogen, linear or branched C₁-C₁₈ alkyl, linear or branched C₂-C₁₈ alkenyl or alkynyl, and 1-(4-aminocyclohexyl) methylene;
Y, Z and W are independently selected in a first group consisting of hydrogen, linear or branched C₁-C₁₈ alkyl, linear or branched C₂-C₁₈ alkenyl or alkynyl, or else in a second group consisting of:

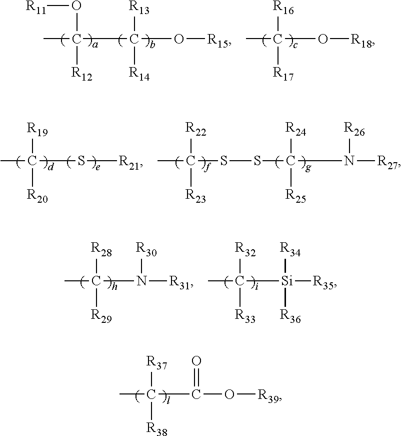

and —R₄₀
wherein R₁₁-R₃₉ are independently selected in the group consisting of: hydrogen, linear or branched C₁-C₁₈ alkyl, linear or branched C₂-C₁₈ alkenyl or alkynyl, aryl, linear or branched C₁-C₂₂ alkyl-aryl, linear or branched C₂-C₂₂ alkenyl-aryl, linear or branched C₂-C₂₂ alkynyl-aryl, heteroaryl and carboxyl;
R₄₀ can be:

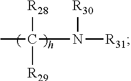

R₃₄-R₃₆ can independently be: —OCH₂—CH₃ or —OCH₃;
R₃₈ can be —CH₂—SH, or —CH₂—CH₂—S—CH₃;
and wherein e is an integer of from 1 to 4, and a-d f-l are, independently of one another, integers of from 1 to 12.

### BRIEF DESCRIPTION OF THE FIGURES

The description is presented here with reference to the appended drawings, provided solely for illustrative purposes and not limiting the invention.

FIG. 1: ¹H NMR spectrum of the crude reaction mixture comprising 2,5-hexanedione obtained from the reaction of 2,5-dimethylfuran according to the first step of the process according to the present invention, as described in example 6;

FIG. 2: ¹H NMR spectrum of the crude reaction mixture comprising serinol-pyrrole obtained according to the second step of the process according to the present invention, as described in example 6.

### DETAILED DESCRIPTION OF THE INVENTION

In a first aspect, the present invention relates to a process for the preparation of a diketone of formula (II) according to the following scheme (1)

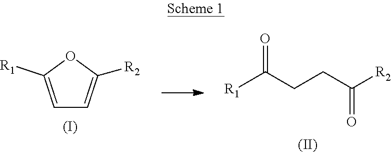

wherein R₁-R₂ are independently selected in the group consisting of hydrogen, a linear or branched C₁-C₁₈ alkyl group, a linear or branched C₂-C₁₈ alkenyl or alkynyl group, an alkyl-aryl group with linear or branched C₁-C₁₈ alkyl, an alkenyl-aryl group with linear or branched C₂-C₁₈ alkenyl, an alkynyl-aryl group with linear or branched C₂-C₁₈ alkynyl, aryl, and heteroaryl; and wherein said process comprises the phases of:
a) preparing a mixture composed of: water, an organic or inorganic Brønsted-Lowry acid having a pKₐ value lower than 3.5 and the one or more conjugate bases whereof have a standard reduction potential value lower than 0.5 volts measured under acidic conditions, and a furan of formula (I); and
b) stirring the mixture, optionally heating;
wherein in the mixture prepared in phase a) the molar ratio between water and furan of formula (I) is at least 1, preferably of from 1 to 4, and wherein said organic or inorganic Brønsted-Lowry acid is present in an amount corresponding to at least 2 mol % with respect to the amount of furan of formula (I).

As will be explained in more detail below, careful selection of the parameters and of the reaction conditions of the process described above makes it possible to obtain diketones, optionally substituted in the 2 and/or 5 positions, in high yields, preferably greater than or equal to 90%, and provides for simple work-ups. Preferably, the process according to the present invention does not require further purification steps.

According to the present invention, the mixture of phase a) does not contain other elements apart from: water, a Brønsted-Lowry acid as defined above, and a furan of formula (I). In other words, in the present description and in the appended claims, the expression “composed of”, referred to the mixture of phase a), indicates that said mixture consists exclusively of the above-mentioned elements, and does not provide for the addition of, for example, organic solvents.

Preferably, the furans optionally substituted at the positions 2 and/or 5 useful for the purposes of the present invention are represented by the formula (I) shown above, wherein the groups R₁-R₂ are independently selected in the group consisting of hydrogen, a linear or branched C₁-C₁₀ alkyl group, a linear or branched C₂-C₁₀ alkenyl or alkynyl group, an alkyl-aryl group with linear or branched C₁-C₁₀ alkyl, an alkenyl-aryl group with linear or branched C₂-C₁₀ alkenyl, an alkynyl-aryl group with linear or branched C₂-C₁₀ alkynyl, aryl, and heteroaryl. Still more preferably, the groups R₁-R₂ are independently selected in the group consisting of hydrogen and a linear or branched C₁-C₁₈ alkyl group. In particularly preferred embodiments, the groups R₁-R₂ are independently selected in the group consisting of hydrogen and a linear or branched C₁-C₆ alkyl group.

Particularly preferred examples of the aforesaid aryl groups are: phenyl, naphthyl and substituted derivatives thereof.

Particularly preferred examples of the aforesaid heteroaryl groups are: pyridyl, furanyl, thiophenyl, pyrrolyl, the respective benzo-fused groups and substituted derivatives.

Preferably, in the mixture prepared in phase a) of the process according to the present invention the molar ratio between water and the furan of formula (I) is of from 1 to 3, still more preferably of from 1 to 2.

It is indeed necessary to use at least a stoichiometric amount of water to obtain the conversion of the selected furan of formula (I) into the compound of formula (II): in fact, using less than 1 equivalent of water no conversion is obtained. On the other hand, using a water/furan molar ratio higher than 1, and preferably of from 1 to 3, even more preferably of from 1 to 2, it is possible to obtain high conversion yields without the formation of any byproduct.

As described above, the organic or inorganic Brønsted-Lowry acids useful for the purposes of the present invention are selected from those having a pKₐ value lower than 3.5, preferably lower than 3, and one or more conjugate bases whereof have a standard reduction potential value lower than 0.5 volts measured under acidic conditions.

As known in the art, a Brønsted-Lowry acid is defined as a substance capable of acting as a source of protons.

The pKₐ and standard reduction potential values correspond to those deriving from measurements performed in water under standard conditions (as stated, for example, in: CRC Handbook of Chemistry and Physics, 97^{th} edition, 2016-2017, chapter 5, pages 78-84 and 87-97). In particular, in the case wherein such acid has several conjugate bases, all these species should have a standard reduction potential value lower than 0.5 volt, when measured under acidic conditions. When there are several redox pairs associated with one and the same conjugate base, all these species should have a standard reduction potential value lower than 0.5 volt, when measured under acidic conditions.

In particular, the organic or inorganic Brønsted-Lowry acids useful for the purposes of the present invention are for example: halohydric acids, in particular hydrochloric acid, hydrobromic acid, hydrofluoric acid, and hydriodic acid; sulfuric acid, trifluoroacetic acid, chloroacetic acid, phosphoric acid, phosphorous acid, sulfurous acid, methanesulfonic acid, citric acid, oxalic acid, triflic acid (H[CF₃SO₃]), and fluoroantimonic acid (H[SbF₆]).

Such acids are preferably selected from the strong organic or inorganic Brønsted-Lowry acids, one or more conjugate bases whereof have a standard reduction potential value lower than 0.5 volt, when measured under acidic conditions. Thus, still more preferably, the strong organic or inorganic Brønsted-Lowry acids are selected from HCl, H₂SO₄, HBr, and methanesulfonic acid.

Advantageously, in the mixture prepared in phase a) of the process according to the present invention, the organic or inorganic Brønsted-Lowry acid is present in an amount corresponding to at least 2 mol % with respect to the amount of furan of formula (I), preferably to at least 2.5 mol % or 3 mol %, still more preferably to at least 3.5 mol % or 4 mol %.

In embodiments, the amount of organic or inorganic Brønsted-Lowry acid is of from 2 mol % to 25 mol % with respect to the amount of furan of formula (I), preferably of from 2 mol % to 20 mol %, or of from 2 mol % to 15 mol %. Still more preferably, the amount of organic or inorganic Brønsted-Lowry acid, with respect to the amount of furan of formula (I), is in the range of 2.5-25 mol %, or 3-25 mol %, or 3.5-25 mol %, or 4-25 mol %, or 2.5-20 mol %, or 3-20 mol %, or 4-20 mol %, or 2.5-15 mol %, or 3-15 mol %, or 3.5-15 mol %, or 4-15 mol %.

According to the present invention, in phase b) the mixture is stirred, optionally heating, to obtain the compound of formula (II). In other words, once the mixture in phase a) has been prepared, it is stirred, optionally heating, in phase b) without there being further, previous or subsequent, phases leading to the obtainment of the desired compound.

In phase b) of the process according to the present invention, the mixture can be brought to and/or maintained at a temperature of from 20° C. to 100° C., preferably of from 20° C. to 80° C., still more preferably of from 20° C. to 60° C. In particularly preferred embodiments, the temperature is of from 20° to 55° C.

Advantageously, when the reaction is performed at a temperature of from 75° C. to 100° C. it is possible to obtain the diketone of formula (II) with very short reaction times, of a few hours, hence rendering the process particularly rapid and advantageous. Preferably, in this case the amount of acid used is of from 2 mol % to 5 mol %, or 2.5 mol % to 5 mol %, preferably of from 3 mol % to 5 mol %, or 3.5 mol % to 5 mol %, still more preferably of from 4 mol % to 5 mol %: the use of the least amount of acid in fact makes it possible to perform the reaction at high temperatures avoiding the formation of oligomeric by-products.

However, in a particularly preferred embodiment, the reaction is performed at a temperature of from 20° C. to 60° C., still more preferably of from 20° C. to 55° C.: in this way it is possible to obtain the diketone of formula (II) in high yields, for example over 90%, without any formation of oligomeric by-products in a wide range of amount of acid used. This makes it possible to avoid purification steps, rendering the process particularly efficient and advantageous.

Under the optimal conditions defined for the process according to the present invention, the reaction proceeds with high conversion percentages of the furan of formula (I) to the diketone of formula (II). According to a preferred embodiment, the reaction has conversion percentages greater than or equal to 80%, preferably greater than or equal to 90%, still more preferably greater than or equal to 95%.

Consequently, for the reasons presented above, it is possible to obtain the diketone of formula (II) in yields greater than or equal to 80%, preferably greater than or equal to 90%, still more preferably greater than or equal to 95%.

Owing to the high conversion percentages, in the embodiments wherein the molar ratio between water and the furan of formula (I) is 1, at the end of the reaction the water used will be completely consumed, hence it is possible to obtain the diketone of formula (II) without performing any further step. Alternatively, in the embodiments wherein the molar ratio between water and the furan of formula (I) is greater than 1 it is possible to obtain the diketone of formula (II) by simply removing the residual water by techniques known in the art, such as for example evaporation, distillation, filtration, etc.

Although not necessary, there can further be provided, also depending on the subsequent use of the diketone of formula (II) or on the conversion percentages obtained, a purification step by techniques known in the art, such as for example crystallization, chromatographic separation, etc.

According to a further embodiment, the process according to the present invention comprises, after the phase b) as defined above, the addition of a primary amine R₂—NH₂ (III). The mixture obtained, comprising the amine, is stirred and optionally heated, to obtain a pyrrole derivative of formula (IV) according to the following synthetic scheme (2)

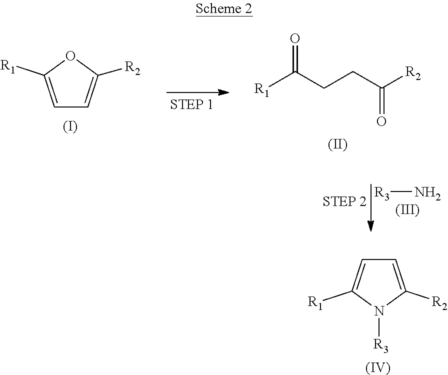

wherein, in the formulae (I)-(IV), the groups R₁ and R₂ have the meanings previously defined and R₃ is selected in the group comprising:

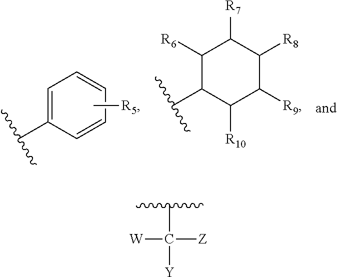

wherein
R₅ is hydrogen, alkyl, aryl, benzyl, amine, alkylamine, arylamine, benzylamine or aminoaryl;
R₆-R₁₀ are independently selected in the group consisting of: hydrogen, linear or branched C₁-C₁₈ alkyl, linear or branched C₂-C₁₈ alkenyl or alkynyl and 1-(4-aminocyclohexyl) methylene;
Y, Z and W are independently selected in a first group consisting of hydrogen, linear or branched C₁-C₁₈ alkyl, linear or branched C₂-C₁₈ alkenyl or alkynyl, or else in a second group consisting of:

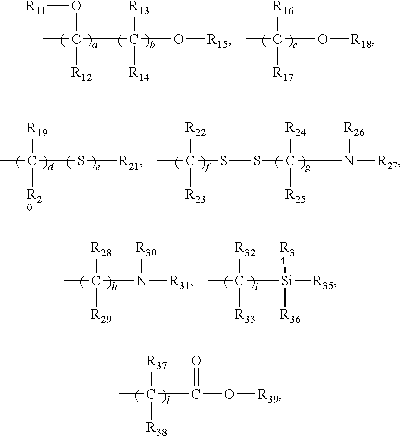

and —R₄₀
wherein R₁₁-R₃₉ are independently selected in the group consisting of: hydrogen, linear or branched C₁-C₁₈ alkyl, linear or branched C₂-C₁₈ alkenyl or alkynyl, aryl, linear or branched C₁-C₂₂ alkyl-aryl, linear or branched C₂-C₂₂ alkenyl-aryl, linear or branched C₂-C₂₂ alkynyl-aryl, heteroaryl and carboxyl;
R₄₀ can be:

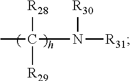

R₃₄-R₃₆ can independently be: —OCH₂—CH₃ or —OCH₃;
R₃₈ can be —CH₂—SH, or —CH₂—CH₂—S—CH₃;
and wherein e is an integer of from 1 to 4, and a-d f-l are, independently of one another, integers of from 1 to 12.

According to an embodiment, the diketone of formula (II) obtained by phase b) is used for the second synthetic step in the presence of the amine without the need for any isolation or purification, simply by adding the amine to the reaction mixture obtained at the end of phase b). According to this implementation, the conversion percentage of the furan (I) to the corresponding diketone (II), before the addition of the amine, can be measured and is preferably greater than or equal to 90%. This conversion can be monitored for example by GC-MS, ¹H-NMR, or ESI-MS.

According to this embodiment, the second step described above comprises the following phases:
c) adding a primary amine R₃—NH₂ (III) to the mixture obtained from phase b) as defined above for the conversion of the diketone of formula (II) into a pyrrole derivative of formula (IV) according to the synthetic scheme (2):

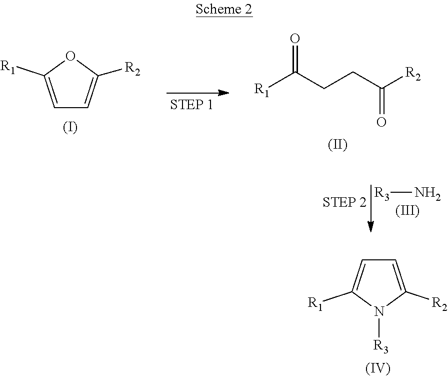

wherein R₁, R₂ and R₃ are as previously defined; and
d) stirring the mixture, optionally with heating.

The amine is preferably added in an equimolar amount with respect to the furan initially present, but it can also advantageously be added in a molar ratio of from 0.8 to 1.5 with respect to the furan. Still more preferably, the amine is added to the mixture in a molar ratio of from 0.8 to 1.2 with respect to the furan of formula (I).

Preferably, the primary amine R₃—NH₂ (III) is selected in the group consisting of: alkylamines with linear or branched C₁-C₁₈ alkyl and optionally substituted with one or more —OH groups, and alkyl-arylamines with linear or branched C₁-C₁₈ alkyl.

In the particularly preferred embodiments, the primary amine R₃—NH₂ (III) is selected in the group consisting of: serinol, isoserinol, benzylamine, alkylamines with linear or branched C₁-C₁₀ alkyl, and alkanolamines with linear or branched C₁-C₁₀ alkyl.

Phase d) of the process for obtaining the pyrrole derivative of formula (IV) can be performed at a temperature of from 20° C. to 200° C. In particular, the temperature is selected with regard to the preselected amine: when very reactive amines are used, for example amines bearing electron donating substituents on the nitrogen, the reaction already proceeds rapidly at room temperature, while when deactivated amines are used, such as for example amines bearing electron attracting substituents on the nitrogen, raising the temperature makes it possible to obtain the pyrrole derivative more quickly.

The process according to the present invention, according to the further implementation described above, makes it possible to obtain pyrrole derivatives of formula (IV) in high yields, preferably greater than or equal to 80%, more preferably greater than or equal to 90%. In particular, it has been found that under the conditions identified in phases a) and b) the yields obtained are very high and no isolation of the intermediate product is necessary.

In the embodiment wherein the amine is added after phase b) without separating the diketone (II) obtained from the reaction mixture, the pyrrole derivatives of formula (IV) are obtained from furans of formula (I) in a so-called “one pot two steps” process. In fact, at the end of the phase b) described above, the primary amine of formula (III) is added without performing any processing or purification of the diketone. This is possible thanks to the high conversion percentages obtained in the first step of the process, to the absence of reaction by-products and the fact that the reaction is performed in water and in presence of an acid, conditions compatible with the subsequent addition of the amine for the reaction of the second step of the process.

Alternatively, however, it is possible to envisage isolating, and optionally purifying, the diketone of formula (II) obtained at the end of phase b), using the product directly in the subsequent step 2.

In further embodiments, the process can comprise a step of formation of an adduct between the pyrrole derivative of formula (IV) obtained as described above and a sp² hybridized carbon allotrope, this step can for example comprise the following phases e)-g):
e) forming a mixture comprising said pyrrole derivative of formula (IV) and at least one sp² hybridized carbon allotrope;
f) supplying energy to the mixture obtained in phase e), obtaining an adduct, and
g) optionally separating the adduct obtained.

Preferably the sp² hybridized carbon allotropes are selected in the group consisting of graphene, nanographite, preferably consisting of few graphene layers (from a few units to about ten), graphite, fullerene, nanotoroids, nanocones, graphene nanoribbons, single-walled or multi-walled carbon nanotubes, and carbon black, also called lampblack.

More preferably, the sp² hybridized carbon allotropes are selected in the group consisting of carbon black, graphene, graphite, high surface area graphite, single-walled or multi-walled carbon nanotubes, and mixtures thereof.

The sp² hybridized carbon allotrope can be selected from single-walled or multi-walled carbon nanotubes, carbon black and mixtures thereof.

The sp² hybridized carbon allotrope preferably contains functional groups selected in the group comprising:
oxygenated groups, preferably hydroxyls and epoxides;
groups containing carbonyls, preferably aldehydes, ketones and carboxylic acids;
groups containing nitrogen atoms, preferably amines, amides, diazonium salts and imines;
groups containing sulfur atoms, preferably sulfides, disulfides, mercaptans, sulfones, and sulfinic and sulfonic groups.

The sp² hybridized carbon allotrope can be added directly to the mixture obtained in phase d) when the conversion of the diketone of formula (II) to the pyrrole derivative of formula (IV) is greater than or equal to 80%, preferably greater than or equal to 90%.

The mixture with the sp² hybridized carbon allotrope can be prepared by simple stirring, mechanical or magnetic, or else by sonication, and the water removed by any suitable method known in the art, such as for example evaporation under vacuum, spray-drying, etc.

The energy transfer of phase f) is performed with the aim of improving the interaction between the pyrrole derivative of formula (IV) and the carbon allotrope. In the absence of energy transfer, that interaction would be weaker and could lead to the partial release of the pyrrole derivative of formula (IV) from the carbon allotrope.

The forms of energy which can be transferred to the mixture to contribute to the formation of the adduct are: mechanical energy, thermal energy, photons, or a combination of two or more of these forms of energy.

Preferably, the thermal energy is supplied at a temperature of from 50 to 180° C., for example for a time of from 15 to 360 minutes.

Preferably the mechanical energy is supplied for a time of from 1 to 360 minutes.

Preferably the energy by irradiation with photons is supplied at a wavelength of from 200 to 380 nm, for example for a time of from 30 to 180 minutes.

### EXPERIMENTAL PART

### Materials

All the reagents and solvents used are commercially available and were obtained and used without further purification.

In particular:

| | | | |
|---|---|---|---|
| 2,5-dimethylfuran | Aldrich | Serinol | Bracco |
| H₂SO₄ (95-97%) | Aldrich | Isoserinol | Bracco |
| HNO₃ (69.5% by weight) | Carlo Erba | Ethanolamine | Aldrich |
| HBr (48% by weight) | Fluka | Hexylamine | Merk |
| HCl (37% by weight) | Carlo Erba | Benzylamine | Aldrich |
| CH₃COOH | Aldrich | Methylamine | Aldrich |

### EXAMPLES

### Example 1: General Procedure for the Synthesis of 2,5-hexanedione from 2,5-dimethylfuran (Step 1)

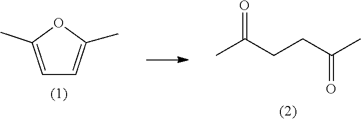

Water, the preselected acid and 2,5-dimethylfuran (1) are poured in sequence into a round-bottomed flask fitted with a magnetic stirrer. The flask is placed in an oil bath maintained at the desired temperature and stirred magnetically (300 rpm).

The dark brown liquid which is obtained is cooled to room temperature and can be analyzed by ¹H NMR and GC-MS.

### Example 2: Optimization of the Reaction Conditions (Time and Temperature)

The procedure described in Example 1 was followed using a 1:1 ratio in moles of water and 2,5-dimethylfuran, and 4 mol %, with respect to the 2,5-dimethylfuran, of H₂SO₄ as the preselected acid. The temperature of the mixture was varied in order to assess the optimal conditions. The reaction was monitored by GC-MS and ¹H-NMR and stopped on reaching a constant amount of product.

The results obtained are summarized in the following table 1.

TABLE 1
DMF/H₂O = 1:1; acid = H₂SO₄ 4 mol %

| T(° C.) | t (h) | Conversion (%) | Yield (%) | Viscosity |
|---|---|---|---|---|
| r.t.^{a} | 24 | 90 | 90 | No |
| 50 | 24 | 95 | 95 | No |
| 75 | 8 | 95 | 95 | Yes |
| 100 | 3 | 95 | 95 | Yes |

^{a}r.t. = room temperature

When the temperature is increased, the reaction proceeds more rapidly and for example a conversion higher than 90% can already be obtained after 3 hours at 100° C. At the same time, however, there is significant formation of undesired oligomeric by-products revealed by a progressive increase in the viscosity of the reaction mixture. The formation of these by-products would render necessary a phase of work-up and purification of the reaction mixture before being able to proceed to a subsequent synthetic step.

For this reason, a temperature of 50° C. and a reaction time of 24 hours were selected as optimal for the subsequent studies, which make it possible to obtain optimal conversion and yield without the formation of any by-product.

### Example 3: Optimization of the Reaction Conditions (Amount of Acid)

The procedure described in Example 1 was followed using a 1:1 ratio in moles of water and 2,5-dimethylfuran, and H₂SO₄ as the preselected acid. The amount of acid used was varied to identify the optimal conditions and is indicated with respect to the amount of 2,5-dimethylfuran used.

For samples 1-5, the reaction was performed at 50° C., stopped after 24 hours and analyzed by ¹H NMR.

For samples 6-7, the reaction was instead performed at 100° C., monitored by ¹H NMR and stopped on reaching a constant amount of product.

The results obtained are summarized in the following table 2.

TABLE 2
DMF/H₂O = 1:1; acid = H₂SO₄

| Sample | T (° C.) | t (h) | H₂SO₄ (mol %) | Yield (%) |
|---|---|---|---|---|
| 1 | 50 | 24 | 0.9 | 13 |
| 2 | 50 | 24 | 1.7 | 17 |
| 3 | 50 | 24 | 4 | 95 |
| 4 | 50 | 24 | 15 | 95 |
| 5 | 50 | 24 | 21 | 95 |
| 6 | 100 | 4 | 4 | 90 |
| 7 | 100 | o/n^{a} | 0.04 | 28 |

^{a}o/n = one night (overnight)

As is clear from the table, performing the reaction at a temperature of 50° C. and using up to 1.7 mol % of sulfuric acid, even after 24 hours the yield remains below 20% (see samples 1-2). Conversely, using already 4 mol % of sulfuric acid after 24 hours 2,5-hexanedione is obtained in a yield of 95% and without the formation of any by-product.

Surprisingly, it was possible to perform the reaction at 100° C. without having the formation of any by-product using 4 mol % of sulfuric acid. In this manner, 2,5-hexanedione was obtained in a yield of 90% after only 4 hours of reaction.

Using instead a minimal amount of sulfuric acid at 100° C. (see sample 7) the yield was only 28%.

### Example 4: Optimization of the Reaction Conditions (Amount of Water)

The procedure described in Example 1 was followed using 15 mol %, calculated with respect to the amount of 2,5-dimethylfuran used, of H₂SO₄ as the preselected acid, and performing the reaction at 50° C. for 24 hours. The amount of water used, hence the ratio between 2,5-dimethylfuran and water, was varied to assess the optimal conditions.

The results obtained are summarized in the following table 3.

TABLE 3
DMF 1 eq.; acid = H₂SO₄ 15 mol %, T = 50° C., t = 24 h

| H₂O (eq.) | Yield (%) |
|---|---|
| <1 | — |
| 1 | 95 |
| 1.5 | 98 |
| 2.0 | 99 |
| 3.0 | 97 |

As is clear from the results presented in the table, it is necessary to use at least a stoichiometric amount of water to obtain the conversion of the 2,5-dimethylfuran to 2,5-hexanedione: indeed, on using less than 1 equivalent of water, no conversion is obtained.

Conversely, on using a water/DMF molar ratio greater than 1 it is possible in every case to obtain a conversion yield greater than or equal to 95% without the formation of any by-product.

### Example 5: Optimization of the Reaction Conditions (Type of Acid)

The procedure described in Example 1 was followed using a 1:1 ratio in moles of water and 2,5-dimethylfuran, 4 mol % of acid, calculated with respect to the amount of 2,5-dimethylfuran used, performing the reaction at 50° C. for 24 hours. Various types of acids were used to investigate their effect on the course of the reaction.

The results obtained are summarized in the following table 4.

TABLE 4
DMF/H₂O = 1:1; acid = 4 mol %; T = 50° C., t = 24 h

| Sample | Acid | Yield (%) |
|---|---|---|
| 1 | H₂SO₄ | 95* |
| 2 | HCl | 97 |
| 3 | HBr | 97 |
| 4 | HNO₃ | <5 (by-products) |
| 5 | CH₃COOH | — |
| 6 | ZnO | — |

*see sample 3 (Table 2) relating to the experiment described in example 3

The results presented above show that the conversion of 2,5-dimethylfuran to 2,5-hexanedione is obtainable through the use of a Brønsted-Lowry acid having a pKₐ lower than 3.5, and one or more conjugate bases whereof have a standard reduction potential value lower than 0.5 volt, measured under acidic conditions.

Using for example HCl, HBr and H₂SO₄ (samples 1-3), acids which fall into this category, under the conditions presented above, a yield of at least 95% is in fact obtained.

Conversely, using acetic acid (sample 5), having a pKₐ greater than 3.5, just as when using a Lewis acid (zinc oxide, sample 6), which is not a Brønsted-Lowry acid, no conversion was observed.

As regards the reaction with nitric acid (sample 4), the conjugate bases whereof all have a standard reduction potential value greater than 0.5 volt, when measured under acidic conditions, this led to the formation of degradation products, very probably due to the oxidizing action of the nitric acid on the furan ring.

### Example 6: General Procedure for the Synthesis of 2-(2,5-dimethyl-1H-pyrrol-1-yl)-1,3-propanediol (SP) from 2,5-dimethylfuran

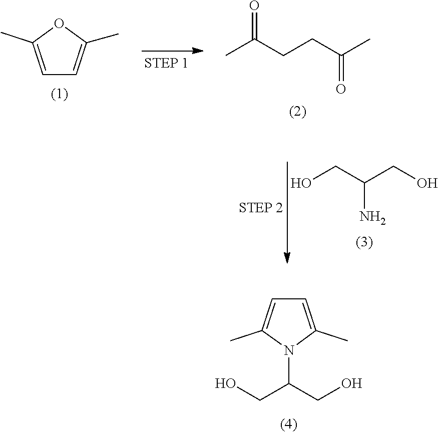

9.39 mmol of water, 4 mol % of sulfuric acid, calculated with respect to the amount of 2,5-dimethyfuran used, and 9.39 mmol of 2,5-dimethylfuran (1) were successively poured into a round-bottomed flask fitted with a magnetic stirrer. The flask was placed in an oil bath at 50° C. and stirred magnetically (300 rpm) for 24 hours. The dark brown liquid obtained was cooled to room temperature and analyzed by ¹H NMR and GC-MS. 2,5-hexanedione (2) was obtained in a yield of 95%.

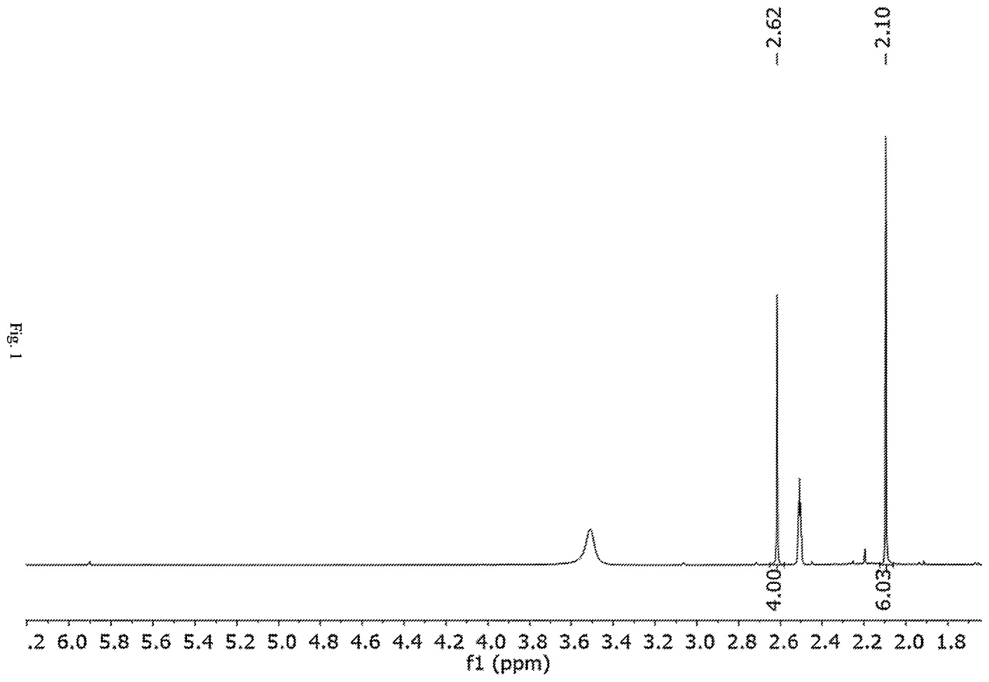

The crude reaction mixture thus obtained was used without any work-up or purification for the second synthetic step. As can be seen from the ¹H-NMR spectrum presented in FIG. 1, in fact, this mixture essentially contains only 2,5-hexanedione (2): the spectrum shows no peak attributable to the starting furan, nor to possible by-products.

After the addition of 9.39 mmol of serinol (3), the mixture was placed at a temperature of 155° C. and stirred for 2.5 hours (300 rpm). The reaction mixture was then cooled to room temperature and analyzed by H NMR and GC-MS. 2-(2,5-dimethyl-1H-pyrrol-1-yl)-1,3-propanediol (SP=serinol pyrrole, 4) was obtained as an amber-red viscous liquid in a yield of 95%.

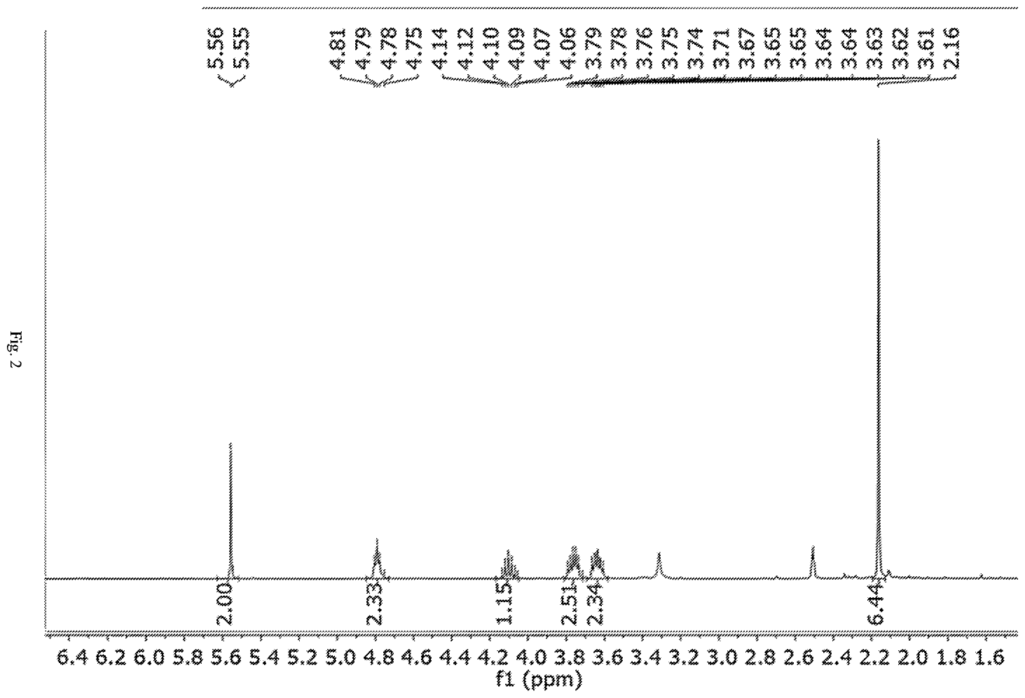

FIG. 2 shows the ¹H NMR spectrum of the product thus obtained: in this case also, it is clear that there are no significant peaks other than those attributable to the serinol pyrrole.

As shown in the following Table 5, it was possible to obtain serinol pyrrole without the formation of any by-product and in an excellent yield even on performing the reaction from 47 mmol of 2,5-dimethylfuran, hence adding 47 mmol of serinol in the second step.

TABLE 5
DMF/serinol = 1/1, T = 155° C., t = 2.5 h

| DMF (mmol) | Serinol (mmol) | Yield (%) |
|---|---|---|
| 9.39 | 9.39 | 95 |
| 47 | 47 | 80 |

### Example 7: Synthesis of Variously Functionalized Pyrroles from 2,5-dimethylfuran

The procedure of example 6 was repeated using other primary amines instead of the serinol. The results obtained are presented in the following Table 6.

The temperature was varied depending on the reactivity of the amine used: aliphatic amines such as methylamine and hexylamine (see samples 3-4) are more reactive and hence the reaction was performed at a lower temperature compared to aromatic amines (for example benzylamine, sample 5) and amines such as ethanolamine which have the doublet on the nitrogen atom less nucleophilic owing to the formation of hydrogen bonds with the hydroxyl in the beta position (sample 2).

In all cases, after 2 hours the reaction mixture was cooled to room temperature and analyzed by ¹H NMR and GC-MS. In all cases the corresponding pyrrole derivatives were obtained in excellent yield (80-94%).

TABLE 6
DMF/amine = 1:1; t = 2 h

| | | | T | Yield |
|---|---|---|---|---|
| Sample | Amine | | (° C.) | (%) |
| 1 | isoserinol | 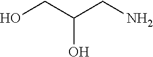 | 50 | 92 |
| 2 | ethanolamine | 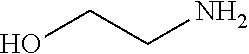 | 155 | 93 |
| 3 | methylamine | Me—NH₂ | r.t.^{a} | 94 |
| 4 | hexylamine | 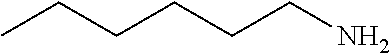 | 60 | 85 |
| 5 | benzylamine | 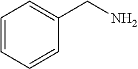 | 100 | 80 |

^{a}r.t. = room temperature

1-19. (canceled)

20. A process for the preparation of a diketone of formula (II) according to the following scheme (1) 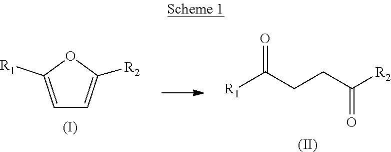
wherein R₁ and R₂ are independently selected in the group consisting of: hydrogen, a linear or branched C₁-C₁₈ alkyl group, a linear or branched C₂-C₁₈ alkenyl or alkynyl group, an alkyl-aryl group with a linear or branched C₁-C₁₈ alkyl group, an alkenyl-aryl group with a linear or branched C₂-C₁₈ alkenyl group, an alkynyl-aryl group with a linear or branched C₂-C₁₈ alkynyl group, aryl, and heteroaryl;
wherein the process comprises the following phases:
a) preparing a mixture composed of: water, an organic or inorganic Brønsted-Lowry acid having a pKₐ value lower than 3.5 and the one or more conjugated bases whereof have a standard reduction potential value lower than 0.5 volt measured under acidic conditions, and a furan of formula (I); and
b) stirring the mixture, and optionally heating;
wherein in the mixture prepared in a), the molar ratio between water and furan of formula (I) is at least 1 and wherein the organic or inorganic Brønsted-Lowry acid is present in an amount corresponding to at least 2 mol % with respect to the amount of furan of formula (I).

21. The process according to claim 20, wherein the molar ratio between water and furan of formula (I) ranges from 1 to 4.

22. The process according to claim 20, wherein the molar ratio between water and furan of formula (I) ranges from 1 to 3.

23. The process according to claim 20, wherein groups R₁ and R₂ are independently selected in the group consisting of: hydrogen, and a linear or branched C₁-C₁₈ alkyl group.

24. The process according to claim 20, wherein the organic or inorganic Brønsted-Lowry acid is selected from the group consisting of: halohydric acids, sulfuric acid, trifluoroacetic acid, chloroacetic acid, phosphoric acid, phosphorous acid, sulphurous acid, methanesulfonic acid, citric acid, oxalic acid, triflic acid (H[CF₃SO₃]), and fluoroantimonic acid (H[SbF₆]).

25. The process according to claim 20, wherein the organic or inorganic Brønsted-Lowry acid is a strong acid, the one or more conjugate bases whereof have a standard reduction potential value lower than 0.5 volt measured under acidic conditions.

26. The process according to claim 25, wherein the strong acid is selected in the group consisting of: HCl, H₂SO₄, HBr, and methanesulfonic acid.

27. The process according to claim 20, wherein in b), the mixture is brought to, kept at, or brought to and kept at a temperature ranging from 20° C. to 100° C.

28. The process according to claim 27, wherein in b), the mixture is brought to, kept at, or brought to and kept at a temperature ranging from 20° C. to 80° C.

29. The process according to claim 28, wherein in b), the mixture is brought to, kept at, or brought to and kept at a temperature ranging from 20° C. to 60° C.

30. The process according to claim 20, wherein the organic or inorganic Brønsted-Lowry acid is present in an amount ranging from 2 mol % to 25 mol % with respect to the amount of furan of formula (I).

31. The process according to claim 30, wherein the organic or inorganic Brønsted-Lowry acid is present in an amount ranging from 2 mol % to 15 mol % with respect to the amount of furan of formula (I).

32. The process according to claim 20, wherein the organic or inorganic Brønsted-Lowry acid is present in an amount ranging from 2 mol % to 5 mol % with respect to the amount of furan of formula (I) and in b), the mixture is brought to and kept at a temperature ranging from 75° C. to 100° C.

33. The process according to claim 20, wherein, after b), further comprising adding a primary amine R₃—NH₂ (III) and stirring the mixture, and optionally heating to obtain a pyrrole derivative of formula (IV) according to the following synthetic scheme (2): 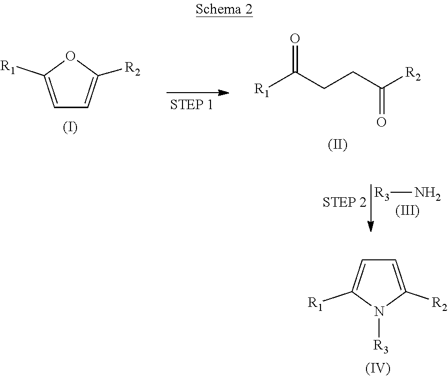
wherein R₁ and R₂ are independently selected in the group consisting of: hydrogen, a linear or branched C₁-C₁₈ alkyl group, a linear or branched C₂-C₁₈ alkenyl or alkynyl group, an alkyl-aryl group with a linear or branched C₁-C₁₈ alkyl group, an alkenyl-aryl group with a linear or branched C₂-C₁₈ alkenyl group, an alkynyl-aryl group with a linear or branched C₂-C₁₈ alkynyl group, aryl, and heteroaryl, and wherein R₃ is selected from the group consisting of: 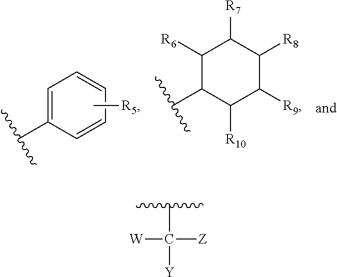
wherein
R₅ is hydrogen, alkyl, aryl, benzyl, amine, alkylamine, arylamine, benzylamine, or aminoaryl;
R₆-R₁₀ are independently selected in the group consisting of: hydrogen, linear or branched C₁-C₁₈ alkyl, linear or branched C₂-C₁₈ alkenyl or alkynyl, and 1-(4-aminocyclohexyl) methylene;
Y, Z, and W are independently selected in a first group consisting of: hydrogen, linear or branched C₁-C₁₈ alkyl, linear or branched C₂-C₁₈ alkenyl or alkynyl, or in a second group consisting of: 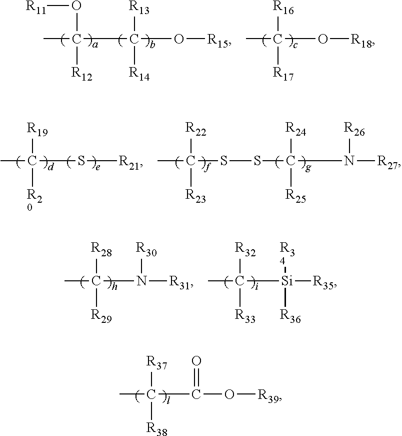
and —R₄₀
wherein R₁₁-R₃₉ are independently selected in the group consisting of: hydrogen, linear or branched C₁-C₁₈ alkyl, linear or branched C₂-C₁₈ alkenyl or alkynyl, aryl, linear or branched C₁-C₂₂ alkyl-aryl, linear or branched C₂-C₂₂ alkenyl-aryl, linear or branched C₂-C₂₂ alkynyl-aryl, heteroaryl and carboxyl;
R₄₀ can be: 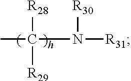
R₃₄-R₃₆ are independently selected in the group consisting of: —OCH₂—CH₃, or —OCH₃;
R₃₈ is: —CH₂—SH, or —CH₂—CH₂—S—CH₃; and
wherein e is an integer ranging from 1 to 4, and a-d f-l are, independently from one another, integers ranging from 1 to 12.

34. The process according to claim 33, wherein the primary amine R₃—NH₂ (III) is added in a molar ratio ranging from 0.8 to 1.5 with respect to the furan of formula (I).

35. The process according to claim 32, wherein the primary amine R₃—NH₂ (III) is selected from the group consisting of alkylamines with linear or branched C₁-C₁₈ alkyl and optionally substituted with one or more —OH groups, and alkyl-aryl-amines with linear or branched C₁-C₁₈ alkyl.

36. The process according to claim 32, wherein the mixture comprising the primary amine R₃—NH₂ (III) is brought to, kept at, or brought to and kept at a temperature ranging from 20° C. to 200° C.

37. The process according to claim 32, wherein prior to adding the primary amine R₃—NH₂ (III) the conversion of the furan of formula (I) to the diketone of formula (II) is equal to or higher than 90%.

38. The process according to claim 37, wherein in a), the molar ratio between water and furan of formula (I) is 1.
